(19) United States
(12) Patent Application Publication
(10) Pub. No.: US 2025/0090142 A1
(43) Pub. Date: Mar. 20, 2025

(54) ULTRASOUND DIAGNOSTIC SYSTEM AND IMAGE PROCESSING APPARATUS

(71) Applicant: FUJIFILM Corporation, Tokyo (JP)

(72) Inventors: Katsunori Asafusa, Tokyo (JP); Hidenao Kubota, Tokyo (JP); Hirohiko Narita, Tokyo (JP)

(21) Appl. No.: 18/826,049

(22) Filed: Sep. 5, 2024

(30) Foreign Application Priority Data
Sep. 14, 2023 (JP) 2023-149575

(51) Int. Cl. A61B 8/08; A61B 8/00

(52) U.S. Cl. CPC A61B 8/5207; A61B 8/4427; A61B 8/463; A61B 8/464; A61B 8/465; A61B 8/56

(57) ABSTRACT

A system capable of generating an ultrasound image with an ultrasound diagnostic apparatus, displaying the ultrasound image with the ultrasound diagnostic apparatus, and performing diagnosis using the ultrasound image with an external device can be provided. An ultrasound diagnostic apparatus acquires Raw data by transmitting and receiving ultrasonic waves, and generates a first ultrasound image by applying first image processing to the Raw data. The Raw data is transmitted as information from the ultrasound diagnostic apparatus to an image processing apparatus. The image processing apparatus generates a second ultrasound image by applying second image processing different from the first image processing to the Raw data. The image processing apparatus displays the second ultrasound image on a display of the image processing apparatus.

### CROSS REFERENCE TO RELATED APPLICATION

The present application claims priority from Japanese Patent Application No. 2023-149571 filed on Sep. 14, 2023, the content of which is hereby incorporated by reference into this application.

### BACKGROUND OF THE INVENTION

### 1. Field of the Invention

The present disclosure relates to an ultrasound diagnostic system and an image processing apparatus.

### 2. Description of the Related Art

In general, an ultrasound diagnostic apparatus acquires Raw data (for example, data to which processing for generating an ultrasound image for display is not applied) by transmitting and receiving ultrasonic waves, applies image processing to the Raw data to generate an ultrasound image, and displays the ultrasound image on a display.

JP2009-112357A discloses a device that generates an ultrasound image based on Raw data as information received by an ultrasound diagnostic apparatus.

### SUMMARY OF THE INVENTION

In general, an ultrasound diagnostic apparatus acquires Raw data by transmitting and receiving ultrasonic waves, applies image processing to the Raw data to generate an ultrasound image, and displays the ultrasound image on a display.

However, in a case in which the ultrasound diagnostic apparatus does not have a function for performing diagnosis (for example, a measurement function or an advanced function such as three-dimensional image processing), the ultrasound diagnostic apparatus cannot perform diagnosis using the function.

It is conceivable that the ultrasound image generated by the ultrasound diagnostic apparatus is transmitted to an external device as information and displayed on the external device. However, a person (for example, a doctor or a family member of a subject) who observes or analyzes the ultrasound image displayed on a display of the external device does not necessarily want to observe or analyze only the ultrasound image generated by the ultrasound diagnostic apparatus. Accordingly, the needs of such a person cannot be met by simply transmitting the ultrasound image displayed on the ultrasound diagnostic apparatus to the external device as information and displaying the ultrasound image on the external device.

An object of the present disclosure is to provide a system capable of generating an ultrasound image with an ultrasound diagnostic apparatus, displaying the ultrasound image with the ultrasound diagnostic apparatus, and performing diagnosis using the ultrasound image with an external device.

According to one aspect of the present disclosure, there is provided an ultrasound diagnostic system comprising: an ultrasound diagnostic apparatus; and an image processing apparatus, in which the ultrasound diagnostic apparatus includes an acquisition unit that acquires Raw data by transmitting and receiving ultrasonic waves, a first image processing unit that generates a first ultrasound image by applying first image processing to the Raw data, a first display controller that displays the first ultrasound image generated by the first image processing unit on a first display of the ultrasound diagnostic apparatus, and a first information transmission unit that transmits the Raw data, to which the first image processing is not applied by the first image processing unit, to the image processing apparatus as information, and the image processing apparatus includes a second information reception unit that receives the Raw data as information, a second image processing unit that generates a second ultrasound image different from the first ultrasound image by applying second image processing different from the first image processing to the Raw data, and a second display controller that displays the second ultrasound image generated by the second image processing unit on a second display of the image processing apparatus.

The second image processing may be image processing that is not realized by the ultrasound diagnostic apparatus.

The second image processing may be image processing of generating an image for which a higher level of calculation capability than in the first image processing is required.

The second image processing may be image processing of generating an image for measurement, and the second ultrasound image may be the image for measurement.

The second display controller may display, on the second display, an image that schematically represents an operation panel provided in the ultrasound diagnostic apparatus and that represents a user interface for receiving input of a parameter for displaying an image used for diagnosis or measurement.

The ultrasound diagnostic apparatus may be a portable ultrasound diagnostic apparatus.

According to another aspect of the present disclosure, there is provided an image processing apparatus comprising: a second information reception unit that receives Raw data as information from an ultrasound diagnostic apparatus that acquires the Raw data through transmission and reception of ultrasonic waves and generates a first ultrasound image by applying first image processing to the Raw data; a second image processing unit that generates a second ultrasound image different from the first ultrasound image by applying second image processing different from the first image processing to the Raw data; and a second display controller that displays the second ultrasound image generated by the second image processing unit on a display.

According to still another aspect of the present disclosure, there is provided an ultrasound diagnostic system comprising: an ultrasound diagnostic apparatus; and an image processing apparatus, in which the ultrasound diagnostic apparatus includes an acquisition unit that acquires Raw data by transmitting and receiving ultrasonic waves, a first image processing unit that generates a first ultrasound image by applying first image processing to the Raw data, a first display controller that displays the first ultrasound image generated by the first image processing unit on a first display of the ultrasound diagnostic apparatus, and a first information transmission unit that transmits the Raw data, to which the first image processing is not applied by the first image processing unit, and first information indicating the first image processing in association with each other to the image processing apparatus as information, and the image processing apparatus includes a second information reception unit that receives the Raw data and the first information, which are associated with each other, as information, a second image processing unit that generates the first ultrasound image by applying the first image processing indicated by the first information to the Raw data and generates a second ultrasound image different from the first ultrasound image by applying second image processing different from the first image processing to the Raw data, and a second display controller that displays the first ultrasound image and the second ultrasound image generated by the second image processing unit on a second display of the image processing apparatus.

The second image processing may be image processing that is not realized by the ultrasound diagnostic apparatus.

The second image processing may be image processing of generating an image for which a higher level of calculation capability than in the first image processing is required.

The second image processing may be image processing of generating an image for measurement, and the second ultrasound image may be the image for measurement.

The second display controller may display the first ultrasound image and the second ultrasound image generated by the second image processing unit side by side on the second display.

The second display controller may display, on the second display, an image that schematically represents an operation panel provided in the ultrasound diagnostic apparatus and that represents a user interface for receiving input of a parameter for displaying an image used for diagnosis or measurement.

The ultrasound diagnostic apparatus may be a portable ultrasound diagnostic apparatus.

According to still another aspect of the present disclosure, there is provided an image processing apparatus comprising: a second information reception unit that receives Raw data and first information indicating first image processing, which are associated with each other, as information from an ultrasound diagnostic apparatus that acquires the Raw data through transmission and reception of ultrasonic waves and generates a first ultrasound image by applying the first image processing to the Raw data; a second image processing unit that generates the first ultrasound image by applying the first image processing indicated by the first information to the Raw data and generates a second ultrasound image different from the first ultrasound image by applying second image processing different from the first image processing to the Raw data; and a second display controller that displays the first ultrasound image and the second ultrasound image generated by the second image processing unit side by side on a display.

According to the present disclosure, it is possible to provide a system capable of generating an ultrasound image with an ultrasound diagnostic apparatus, displaying the ultrasound image with the ultrasound diagnostic apparatus, and performing diagnosis using the ultrasound image with an external device.

### BRIEF DESCRIPTION OF THE DRAWINGS

FIG. 1 is a diagram showing an example of a configuration of an ultrasound diagnostic system according to an embodiment.

FIG. 2 is a diagram showing an example of a configuration of an ultrasound diagnostic apparatus according to the embodiment.

FIG. 3 is a diagram showing an example of a configuration of an image processing apparatus according to the embodiment.

FIG. 4 is a diagram showing an example of a structure of information transmission data.

FIG. 5 is a diagram showing data and a main memory.

FIG. 6 is a diagram showing an example of processing by the ultrasound diagnostic system.

FIG. 7 is a diagram showing an example of processing by a processor of the image processing apparatus.

FIG. 8 is a diagram showing an example of processing by the processor of the image processing apparatus.

FIG. 9 is a diagram showing an example of processing by the processor of the image processing apparatus.

FIG. 10 is a diagram showing a display example of an ultrasound image.

FIG. 11 is a diagram showing a display example of an ultrasound image.

FIG. 12 is a diagram showing a display example of an ultrasound image.

FIG. 13 is a diagram showing a display example of an ultrasound image.

FIG. 14 is a diagram showing a display example of an ultrasound image.

### DESCRIPTION OF THE PREFERRED EMBODIMENTS

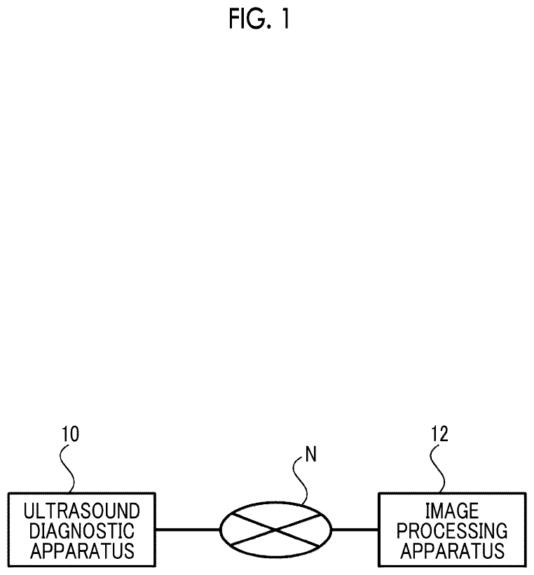

An ultrasound diagnostic system according to an embodiment will be described with reference to FIG. 1. FIG. 1 is a block diagram showing a configuration of the ultrasound diagnostic system according to the embodiment.

The ultrasound diagnostic system includes an ultrasound diagnostic apparatus 10 and an image processing apparatus 12. The ultrasound diagnostic apparatus 10 and the image processing apparatus 12 have a function of communicating with each other via a communication path N.

For example, the communication path N is a network such as the Internet or a local area network (LAN). The communication path N may be formed by wireless communication or may be formed by wired communication. The communication path N may include a high-speed communication path. For example, the high-speed communication path is a wireless communication path using a sixth generation mobile communication system (6G) or a wireless communication path using a next-generation communication system after 6G.

The ultrasound diagnostic apparatus 10 transmits ultrasonic waves into a subject using an ultrasound probe and receives the ultrasonic waves reflected in the subject, thereby generating data representing an inside of the subject. Image processing is applied to the data to generate an ultrasound image representing a tissue or the like inside the subject. The ultrasound diagnostic apparatus 10 may be a portable ultrasound diagnostic apparatus. For example, a tablet-type ultrasound diagnostic apparatus, a portable-type ultrasound diagnostic apparatus, a notebook-type ultrasound diagnostic apparatus, or the like may be used as the ultrasound diagnostic apparatus 10. Of course, the ultrasound diagnostic apparatus 10 may be an ultrasound diagnostic apparatus that is not a portable type.

The data generated by the ultrasound diagnostic apparatus 10 is transmitted as information to the image processing apparatus 12 via the communication path N. For example, in a case in which an information transmission request is transmitted as information from the image processing apparatus 12 to the ultrasound diagnostic apparatus 10, the data is transmitted as information from the ultrasound diagnostic apparatus 10 to the image processing apparatus 12 via the communication path N in response to the request.

The image processing apparatus 12 receives the data from the ultrasound diagnostic apparatus 10 via the communication path N as information. The image processing apparatus 12 generates an ultrasound image by applying image processing to the data. For example, the image processing apparatus 12 is a computer such as a medical image diagnosis support system, a DICOM server, or a medical image analysis system.

For example, the ultrasound diagnostic apparatus 10 generates Raw data by transmitting and receiving the ultrasonic waves. The Raw data is data to which image processing for generating an ultrasound image for display is not applied. The Raw data is transmitted as information from the ultrasound diagnostic apparatus to an image processing apparatus. The image processing apparatus 12 generates an ultrasound image by applying image processing to the Raw data. For example, the ultrasound image is displayed on a display of the image processing apparatus 12.

For example, in a case in which a user of the image processing apparatus 12 issues an instruction to execute the image processing, the image processing apparatus 12 generates the ultrasound image by applying the image processing instructed by the user to the Raw data. For example, the image processing apparatus 12 executes image processing different from the image processing executed by the ultrasound diagnostic apparatus 10. Of course, the image processing apparatus 12 may execute the same image processing as the image processing executed by the ultrasound diagnostic apparatus 10.

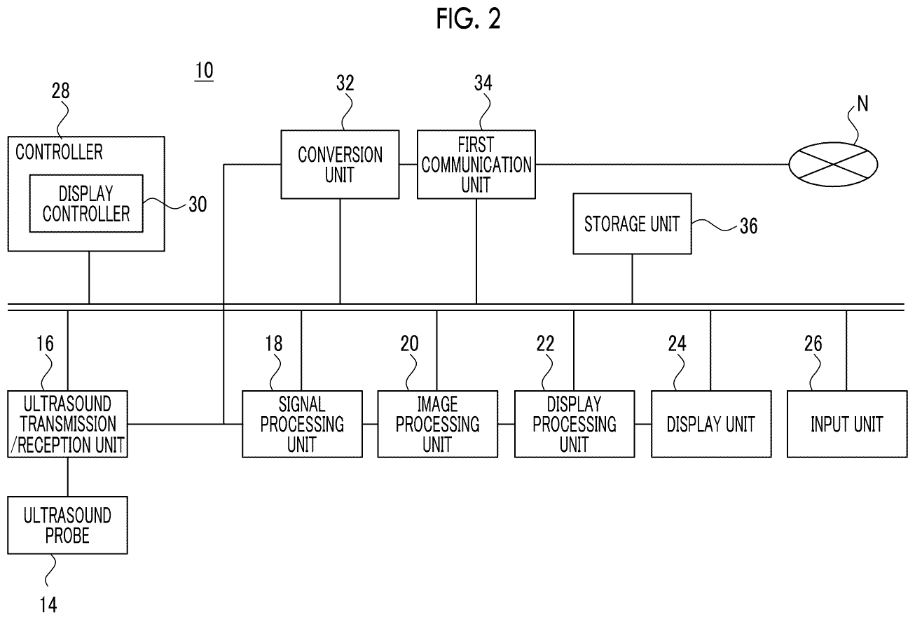

Hereinafter, a configuration of the ultrasound diagnostic apparatus 10 will be described with reference to FIG. 2. FIG. 2 is a block diagram showing an example of the configuration of the ultrasound diagnostic apparatus 10.

The ultrasound diagnostic apparatus 10 includes an ultrasound probe 14, an ultrasound transmission/reception unit 16, a signal processing unit 18, an image processing unit 20, a display processing unit 22, a display unit 24, an input unit 26, a controller 28, a display controller 30, a conversion unit 32, a first communication unit 34, and a storage unit 36.

The ultrasound probe 14 is a device that transmits and receives the ultrasonic waves. For example, the ultrasound probe 14 includes an array transducer. The array transducer is formed by arranging a plurality of ultrasound transducers. An ultrasound beam is formed by the array transducer. In a case in which electronic scanning with the ultrasound beam is repeatedly performed, a scanning surface as an echo data acquisition space is formed for each electronic scanning. In a case in which scanned with the ultrasound beam is performed, an echo data acquisition space is formed. As a scanning method, sector scanning, linear scanning, convex scanning, or the like is used.

In transmitting the ultrasonic waves, the ultrasound transmission/reception unit 16 supplies a plurality of ultrasound transmission signals having a certain delay relationship to the plurality of ultrasound transducers included in the ultrasound probe 14. As a result, a transmission beam of the ultrasonic waves is formed. In receiving the ultrasonic waves, a reflected wave (RF signal) from a living body is received as the ultrasonic wave by the ultrasound probe 14. As a result, a plurality of ultrasound reception signals are output from the ultrasound probe 14 to the ultrasound transmission/reception unit 16. The ultrasound transmission/reception unit 16 forms an ultrasound reception beam by applying phasing addition processing to the plurality of ultrasound reception signals. Data of the ultrasound reception beam is output to the signal processing unit 18. That is, the ultrasound transmission/reception unit 16 forms the ultrasound reception beam by performing delay processing on the ultrasound reception signal obtained from each ultrasound transducer in accordance with a delay processing condition for each ultrasound transducer and performing addition processing on the plurality of ultrasound reception signals obtained from the plurality of ultrasound transducers. The delay processing condition is defined by ultrasound reception delay data indicating a delay time. An ultrasound reception delay data set (that is, a set of delay times) corresponding to the plurality of ultrasound transducers is supplied from the controller 28. The ultrasound transmission/reception unit 16 functions as an ultrasound transmission beam former and an ultrasound reception beam former. For example, the ultrasound transmission/reception unit 16 includes an A/D converter, a detector, an amplification circuit, and the like. The unit that transmits and receives the ultrasonic waves and the unit that realizes the beam former may be configured as separate units. The ultrasound transmission/reception unit 16 corresponds to an example of an acquisition unit.

The signal processing unit 18 applies signal processing to the beam data output from the ultrasound transmission/reception unit 16. For example, the filter processing and the signal processing include detection and amplitude compression such as logarithmic compression. The data to which the signal processing is applied is output to the image processing unit 20.

The image processing unit 20 generates an ultrasound image by applying image processing to the data to which the signal processing is applied. For example, the image processing includes a coordinate transformation function and an interpolation processing function using a digital scan converter (DSC). For example, the ultrasound image is a B-mode image, a color Doppler image, a pulse Doppler image, a strain image, a shear wave elastography image, or the like.

The display processing unit 22 generates a display image by overlaying necessary graphic data on the ultrasound image. The display image is output to the display unit 24. One or a plurality of images are arranged and displayed in a display aspect according to a display mode.

The display unit 24 is a display such as a liquid crystal display or an EL display. The ultrasound image such as the B-mode image is displayed on the display unit 24. The display unit 24 may be a device comprising a display and the input unit 26. For example, a graphic user interface (GUI) may be realized by the display unit 24. In addition, a user interface such as a touch panel may be realized by the display unit 24.

The input unit 26 is a device for the user to input various types of information (for example, a condition required for imaging, a command, and patient information) to the ultrasound diagnostic apparatus 10. For example, the input unit 26 is an operation panel, a switch, a button, a keyboard, a mouse, a track ball, a joystick, or the like.

The controller 28 controls an operation of each unit of the ultrasound diagnostic apparatus 10. The controller 28 includes the display controller 30.

The display controller 30 displays the ultrasound image on the display unit 24. The display controller 30 may display information or an image other than the ultrasound image on the display unit 24.

The conversion unit 32 converts the Raw data into information transmission data. The Raw data is data transmitted as information from the ultrasound diagnostic apparatus 10 to the image processing apparatus 12. For example, the conversion unit 32 generates the information transmission data by adding header data to the Raw data. For example, US header data and IP header data are added to the Raw data as the header data.

The Raw data is data to which processing for generating the ultrasound image for display is not applied. For example, data that is output from the ultrasound transmission/reception unit 16 and to which the signal processing by the signal processing unit 18 is not applied is the Raw data. In this case, the ultrasound transmission/reception unit 16 corresponds to an example of an acquisition unit. Data to which the signal processing by the signal processing unit 18 is applied may be the Raw data. That is, data that is output from the signal processing unit 18 and to which the image processing by the image processing unit 20 is not applied may be the Raw data. In this case, the signal processing unit 18 corresponds to an example of an acquisition unit.

The US header data is metadata (that is, accessory information) added to the Raw data. For example, the US header data includes information on an imaging mode, information on the Raw data, scan information, signal processing information, image processing information, measurement information, frame information, patient information, and the like. The imaging mode is an imaging mode executed by the ultrasound diagnostic apparatus 10 in order to acquire the Raw data. For example, the imaging mode is a B mode (THI), an M mode, a PW mode, a CW mode, a CFI mode, a CFA mode, a TE mode, an SWE mode, or the like. The information on the Raw data includes a data length, the number of samples, an interval, a size, an address, a data type (rectangular coordinate or polar coordinate rθ), and the like. The scan information includes the number of lines, an interval, a size, an address, and a scan type (sector scan, linear scan, or convex scan). The signal processing information is information indicating the signal processing by the signal processing unit 18. The signal processing information includes information on a gain, a filter, or the like. The image processing information is information indicating the image processing by the image processing unit 20. The image processing information includes information on a region of interest (ROI), a filter, or the like. The measurement information includes a parameter related to the measurement executed by the ultrasound diagnostic apparatus 10, and the like. The frame information is information indicating a size, a position, or the like of the ultrasound image. The patient information is information indicating an ID, a gender, an age, or the like of a patient from whom the Raw data is acquired. For example, in a case in which the imaging is performed by the ultrasound diagnostic apparatus 10, each information included in the US header data is input to the ultrasound diagnostic apparatus 10 by the user, such as an examination technician or a doctor, or is input to the ultrasound diagnostic apparatus 10 from an external device, such as a server. Information other than the above-described information may be included in the US header data. The user may designate the information included in the US header data.

The IP header data is accessory information for communication, and includes, for example, information related to an Internet protocol.

The first communication unit 34 is a communication interface including a first information transmission unit and a first information reception unit. The first communication unit 34 has a function of transmitting data as information to an external device and a function of receiving data as information. For example, the first communication unit 34 transmits the Raw data as information to the image processing apparatus 12 via the communication path N. As will be described below, the image processing apparatus 12 generates an ultrasound image by applying image processing to the Raw data transmitted from the ultrasound diagnostic apparatus 10. As another example, the first communication unit 34 transmits the information transmission data as information to the image processing apparatus 12 via the communication path N. As a result, the Raw data and the US header data are transmitted as information from the ultrasound diagnostic apparatus 10 to the image processing apparatus 12.

The storage unit 36 constitutes one or a plurality of storage regions for storing data. For example, the storage unit 36 is a hard disk drive (HDD), a solid state drive (SSD), various memories (for example, RAM, DRAM, or ROM), other storage devices (for example, optical disk), or a combination thereof. For example, the storage unit 36 stores Raw data, ultrasound image data, information indicating imaging conditions, patient information, and the like. The US header data or the information transmission data may be stored in the storage unit 36.

The image processing unit 20 corresponds to an example of a first image processing unit. The image processing by the image processing unit 20 corresponds to an example of first image processing. The ultrasound image generated by applying the first image processing to the Raw data corresponds to an example of a first ultrasound image. The display of the display unit 24 corresponds to an example of a first display. The display controller 30 corresponds to an example of a first display controller. The first communication unit 34 corresponds to an example of a first information transmission unit.

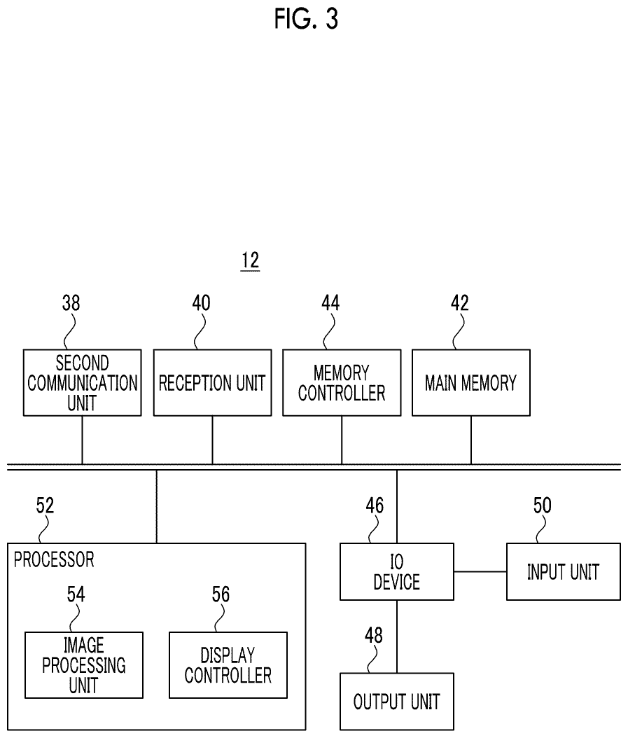

Hereinafter, a configuration of the image processing apparatus 12 will be described with reference to FIG. 3. FIG. 3 is a diagram showing an example of the configuration of the image processing apparatus 12.

The image processing apparatus 12 includes a second communication unit 38, a reception unit 40, a main memory 42, a memory controller 44, an IO device 46, an output unit 48, an input unit 50, and a processor 52.

The second communication unit 38 is a communication interface including a second information transmission unit and a second information reception unit. The second communication unit 38 has a function of transmitting data as information to an external device and a function of receiving data as information. For example, the second communication unit 38 receives the Raw data transmitted as information from the ultrasound diagnostic apparatus 10 via the communication path N, as information. As will be described below, the image processing unit 54 generates an ultrasound image by applying image processing to the Raw data. The image processing executed by the image processing unit 54 is image processing different from the first image processing executed by the ultrasound diagnostic apparatus 10. Of course, the image processing unit 54 may execute the same image processing as the first image processing executed by the ultrasound diagnostic apparatus 10. As another example, the second communication unit 38 receives the information transmission data transmitted as information from the ultrasound diagnostic apparatus 10 via the communication path N, as information. As a result, the second communication unit 38 receives the Raw data as information.

The reception unit 40 receives the information transmission data received as information by the second communication unit 38 and executes a process of storing the information transmission data in the main memory 42.

The main memory 42 constitutes one or a plurality of storage regions for storing data. The main memory 42 is, for example, a hard disk drive (HDD), a solid state drive (SSD), various memories (for example, RAM, DRAM, or ROM), other storage devices (for example, optical disk), or a combination thereof. For example, the information transmission data is stored in the main memory 42. The Raw data and the header data may be separately stored in the main memory 42. The other information may be stored in the main memory 42.

The memory controller 44 controls the main memory 42. For example, the memory controller 44 stores the information transmission data received by the reception unit 40 in the main memory 42. In addition, the memory controller 44 controls the reading of the data from the main memory 42.

The I/O device 46 is connected to the output unit 48 and the input unit 50. For example, various types of information are output to the output unit 48 via the IO device 46 and are input to the image processing apparatus 12 from the input unit 50 via the IO device 46.

The output unit 48 includes a display unit. The display unit is a display such as a liquid crystal display or an EL display. The display unit may be a device comprising a display and the input unit 50. For example, a graphic user interface (GUI) may be realized by the display unit. In addition, a user interface such as a touch panel may be realized by the display unit. The output unit 48 may include a speaker.

The input unit 50 is a device for the user to input various types of information to the image processing apparatus 12. For example, the input unit 50 is an operation panel, a switch, a button, a keyboard, a mouse, a track ball, a joystick, or the like.

The processor 52 includes a controller, an operation unit, a cache, a bus I/F, and the like. The controller controls the image processing apparatus 12. The operation unit performs various operations. In addition, the processor 52 includes an image processing unit 54 and a display controller 56.

The image processing unit 54 generates an ultrasound image by applying image processing to the Raw data. The image processing may include the signal processing described above.

The display controller 56 displays the ultrasound image on a display of the output unit 48. The display controller 56 may display information or an image other than the ultrasound image on the display of the output unit 48.

The image processing unit 54 corresponds to an example of a second image processing unit. The image processing by the image processing unit 54 corresponds to an example of second image processing. The ultrasound image generated by applying the second image processing to the Raw data corresponds to an example of a second ultrasound image. The display of the output unit 48 corresponds to an example of a second display. The display controller 56 corresponds to an example of a second display controller. The second communication unit 38 corresponds to an example of a second information reception unit.

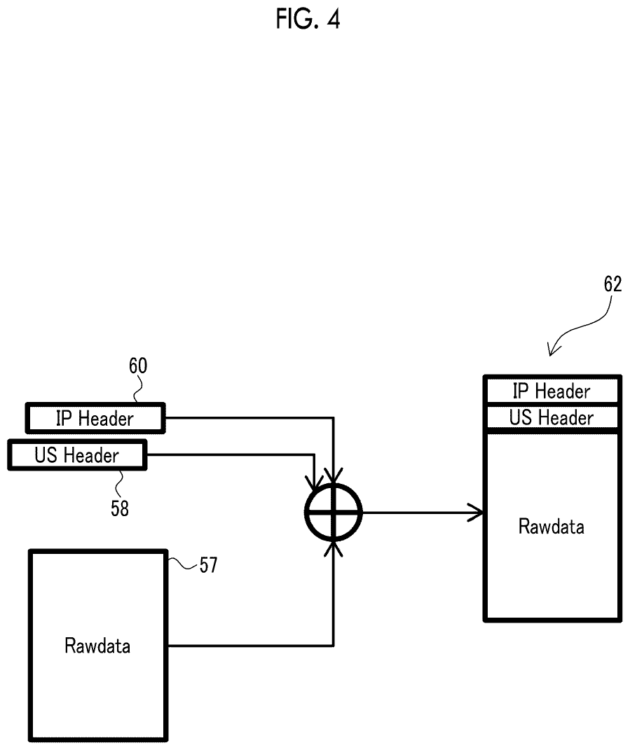

Hereinafter, a structure of the information transmission data will be described with reference to FIG. 4. FIG. 4 shows an example of the structure of the information transmission data.

The ultrasound diagnostic apparatus 10 generates Raw data 57 by transmitting and receiving the ultrasonic waves. For example, the conversion unit 32 generates information transmission data 62 by adding US header data 58 and IP header data 60 to Raw data 57. The information transmission data 62 is transmitted as information from the ultrasound diagnostic apparatus 10 to the image processing apparatus 12.

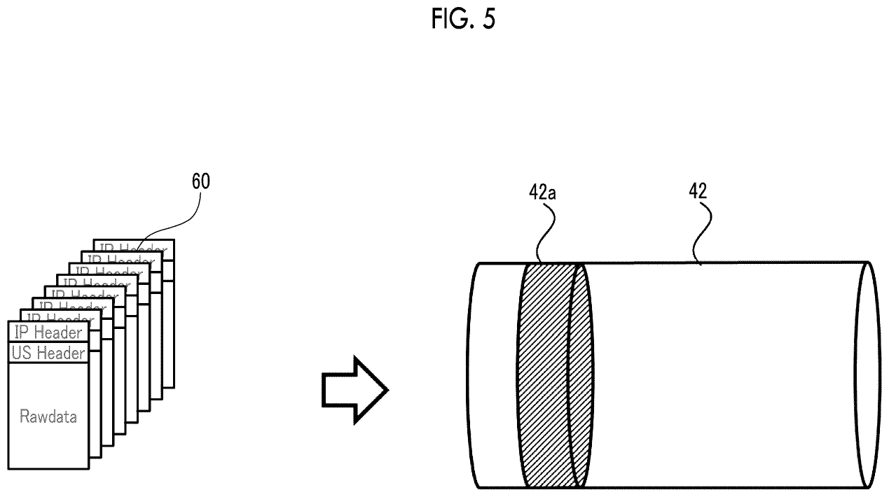

For example, as shown in FIG. 5, the information transmission data 62 is stored in the main memory 42 of the image processing apparatus 12. A storage region 42a for the Raw data is shown in FIG. 5.

For example, the image processing unit 54 of the image processing apparatus 12 reads out the designated Raw data from the main memory 42, and generates an ultrasound image or audio data by applying the operation, the image processing, the audio processing, or the like to the Raw data in accordance with the signal processing or the image processing indicated by the US header data. The image processing unit 54 may convert the measurement information and the patient information included in the US header data into image data. The ultrasound image, the measurement information, and the patient information are displayed on the display of the output unit 48, and the audio data is output from a speaker.

As an example different from the examples shown in FIGS. 4 and 5, the Raw data may be transmitted as information from the ultrasound diagnostic apparatus 10 to the image processing apparatus 12 and stored in the main memory 42 of the image processing apparatus 12. The image processing unit 54 reads out the designated Raw data from the main memory 42, and generates an ultrasound image or audio data by applying the operation, the image processing, the audio processing, or the like to the Raw data in accordance with the designated signal processing or image processing. The signal processing or the image processing applied in this case may be processing different from the signal processing or the image processing applied in the ultrasound diagnostic apparatus 10. That is, the image processing unit 54 may generate an ultrasound image or audio data by applying signal processing or image processing different from the signal processing or the image processing applied in the ultrasound diagnostic apparatus 10, to the Raw data.

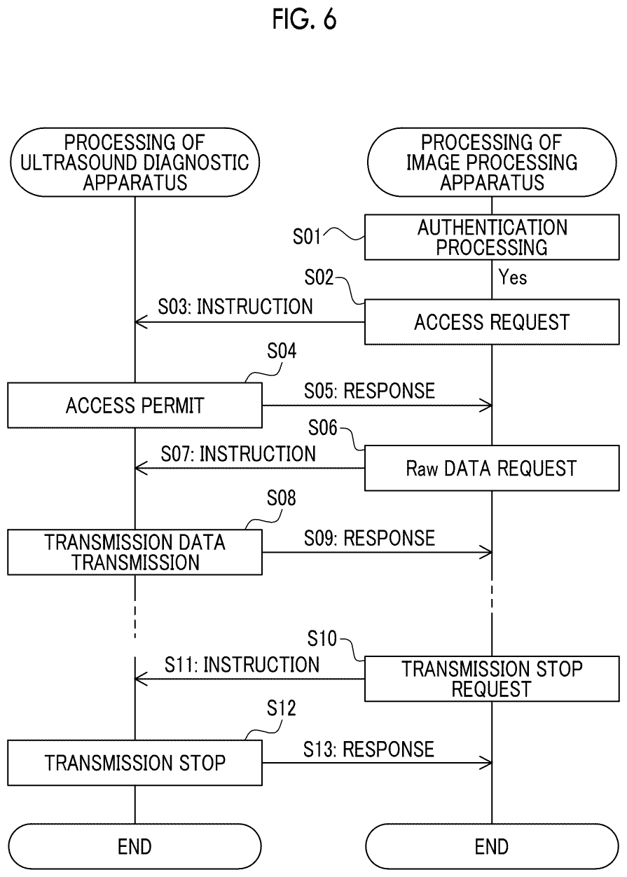

Hereinafter, a flow of processing by the ultrasound diagnostic system will be described with reference to FIG. 6. FIG. 6 shows a flowchart showing the flow of processing by the ultrasound diagnostic system. The following instructions or responses are performed between the first communication unit 34 of the ultrasound diagnostic apparatus 10 and the second communication unit 38 of the image processing apparatus 12 via the communication path N.

First, authentication is performed by the image processing apparatus 12 (S01). For example, the authentication is performed using user personal information, such as a model, a password, a face, or a fingerprint, registered in advance in the image processing apparatus 12. In a case in which the authentication is successful, the processes of step S02 and subsequent steps are executed. In a case in which the authentication fails, the processes of step S02 and subsequent steps are not executed. The authentication does not need to be performed. In this case, the process of step S01 is not performed, and the processes of step S02 and subsequent steps are executed. Hereinafter, the processes of step S02 and subsequent steps will be described.

For example, an instruction screen is displayed on the display of the image processing apparatus 12. In a case in which the user requests access to the ultrasound diagnostic apparatus 10 on the instruction screen (S02), the image processing apparatus 12 issues an instruction to access the ultrasound diagnostic apparatus 10 (S03).

In a case in which the instruction for the access is received, the ultrasound diagnostic apparatus 10 permits the access to the ultrasound diagnostic apparatus 10 (S04) and transmits a response corresponding to the instruction to the image processing apparatus 12 as information (S05). The ultrasound diagnostic apparatus 10 may determine whether or not to permit the access by using the personal information of the user (for example, the personal information input to the image processing apparatus 12).

In a case in which the response is received as information from the ultrasound diagnostic apparatus 10, the image processing apparatus 12 requests the ultrasound diagnostic apparatus 10 to transmit information on the Raw data (S06). The image processing apparatus 12 instructs the ultrasound diagnostic apparatus 10 to transmit the information on the Raw data (S07).

In a case in which the instruction to transmit the information on the Raw data is received, the ultrasound diagnostic apparatus 10 transmits the information transmission data including the Raw data, the US header data, and the IP header data to the image processing apparatus 12 as information (S08).

The ultrasound diagnostic apparatus 10 may include the Raw data being currently acquired by the ultrasound diagnostic apparatus 10 in the information transmission data, and transmit the information transmission data to the image processing apparatus 12 as information. The ultrasound diagnostic apparatus 10 may include the Raw data designated by the user of the image processing apparatus 12 in the information transmission data, and transmit the information transmission data to the image processing apparatus 12 as information. For example, a list of the Raw data acquired by the ultrasound diagnostic apparatus 10 (for example, a list of file names, thumbnail images, and the like) is transmitted as information from the ultrasound diagnostic apparatus 10 to the image processing apparatus 12, and is displayed on the display of the image processing apparatus 12. In a case in which the user of the image processing apparatus 12 designates the Raw data from the list, identification information for identifying the designated Raw data is transmitted as information from the image processing apparatus 12 to the ultrasound diagnostic apparatus 10. The ultrasound diagnostic apparatus 10 transmits information transmission data including the Raw data indicated by the identification information to the image processing apparatus 12 as information.

The information transmission data is transmitted as information to the image processing apparatus 12 as a response to the instruction to transmit the information on the Raw data (S09). The Raw data is stored in the main memory 42 of the image processing apparatus 12. In addition, the image processing apparatus 12 generates an ultrasound image by applying the image processing to the Raw data. The ultrasound image is displayed on the display of the image processing apparatus 12.

After that, in a case in which the user requests the image processing apparatus 12 to stop the transmission of the information on the Raw data (S10), the image processing apparatus 12 instructs the ultrasound diagnostic apparatus 10 to stop the transmission of the information (S11).

In a case in which the instruction to stop the transmission of the information is received, the ultrasound diagnostic apparatus 10 stops the transmission of the information transmission data as information (S12). As a result, the information transmission data is not transmitted as information from the ultrasound diagnostic apparatus 10 to the image processing apparatus 12. A response to the transmission stop of the information is transmitted as information from the ultrasound diagnostic apparatus 10 to the image processing apparatus 12 (S13).

As an example different from the above example, in a case in which the instruction to transmit the information on the Raw data is received, the ultrasound diagnostic apparatus 10 may transmit the Raw data to the image processing apparatus 12 as information (S08). The Raw data is transmitted as information to the image processing apparatus 12 as a response to the instruction to transmit the information on the Raw data (S09). The image processing apparatus 12 generates an ultrasound image by applying image processing to the Raw data. The ultrasound image is displayed on the display of the image processing apparatus 12.

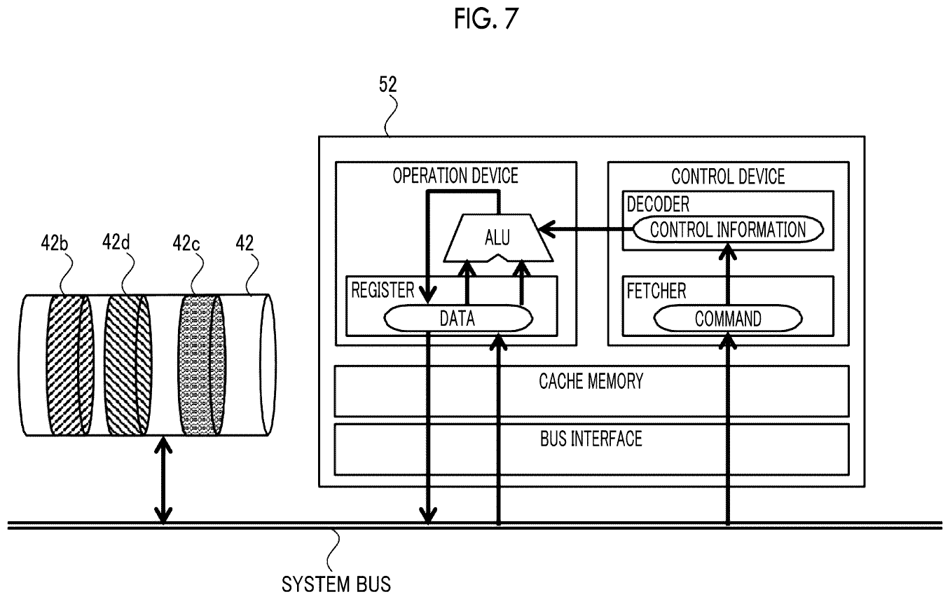

Hereinafter, processing by the processor 52 of the image processing apparatus 12 will be described with reference to FIG. 7.

In a case in which the Raw data is transmitted as information from the ultrasound diagnostic apparatus 10 to the image processing apparatus 12, the Raw data is stored in a storage region 42b of the main memory 42. As another example, in a case in which the transmission data is transmitted as information from the ultrasound diagnostic apparatus 10 to the image processing apparatus 12, the Raw data and the US header data are stored in a storage region 42b of the main memory 42. A command for the image processing is stored in a storage region 42c of the main memory 42. For example, the command for the image processing is input to the image processing apparatus 12 via the input unit 50 and stored in the main memory 42, or is input to the image processing apparatus 12 via the second communication unit 38 and stored in the main memory 42.

For example, the processor 52 includes a bus interface, a cache memory, a control device, and an operation device. The control device includes a fetcher and a decoder. The operation device includes an arithmetic logic unit (ALU) and a register.

The command stored in the storage region 42c is output to the fetcher via a system bus, a bus interface, and a cache memory, and is converted into control information by the decoder. The control information is output to the ALU. The Raw data stored in the storage region 42b is output to the register via the system bus, the bus interface, and the cache memory, and is output to the ALU from the register. The ALU performs an operation on the Raw data based on the control information. The ALU outputs result data generated by the operation to the main memory 42 via the register, the cache memory, the bus interface, and the system bus. The result data is stored in a storage region 42d of the main memory 42. For example, the result data is ultrasound image data.

In the present embodiment, the image processing unit 20 of the ultrasound diagnostic apparatus 10 generates a first ultrasound image by applying the first image processing to the Raw data. The display controller 30 displays the first ultrasound image on the display unit 24.

For example, the ultrasound diagnostic apparatus 10 transmits the Raw data to the image processing apparatus 12. The image processing apparatus 12 receives the Raw data as information. The processor 52 of the image processing apparatus 12 generates a second ultrasound image different from the first ultrasound image by applying second image processing different from the first image processing to the Raw data. The processor 52 displays the second ultrasound image on the display of the output unit 48.

As another example, the ultrasound diagnostic apparatus 10 transmits the Raw data and the first information indicating the first image processing in association with each other to the image processing apparatus 12 as information. For example, the first information is included in the US header data and is transmitted as information to the image processing apparatus 12 in a state of being added to the Raw data. The image processing apparatus 12 receives the Raw data and the first information (for example, the first information included in the US header data), which are associated with each other, as information. The processor 52 of the image processing apparatus 12 generates a first ultrasound image by applying the first image processing indicated by the first information to the Raw data. In addition, the processor 52 generates a second ultrasound image different from the first ultrasound image by applying second image processing different from the first image processing to the Raw data. The processor 52 displays the first ultrasound image and the second ultrasound image side by side on the display of the output unit 48. Of course, the processor 52 may display the first ultrasound image and the second ultrasound image separately on the display rather than displaying the first ultrasound image and the second ultrasound image side by side on the display. The processor 52 may switch the displayed ultrasound image between the first ultrasound image and the second ultrasound image in response to the user's instruction.

For example, the second image processing is image processing that is not realized by the ultrasound diagnostic apparatus 10. That is, the second image processing is image processing realized by a function that is not possessed by the ultrasound diagnostic apparatus 10. For example, the second image processing includes a measurement function, an examination function, or a diagnostic function that is not possessed by the ultrasound diagnostic apparatus 10. As a result, the measurement, the examination, the diagnosis, or the like that could not be performed by the ultrasound diagnostic apparatus 10 can be performed by using the image processing apparatus 12. For example, the second image processing is image processing of generating an image for which a higher level of calculation capability than in the first image processing is required. The second ultrasound image is an image for which a higher level of calculation capability than in the first ultrasound image is required.

For example, the second image processing is image processing of generating a diagnosis image, and the second ultrasound image is an image used for diagnosis. The image processing apparatus 12 executes the second image processing, whereby the user (for example, a doctor) of the image processing apparatus 12 can perform the diagnosis by referring to the second ultrasound image used for the diagnosis. For example, the user such as the doctor can perform diagnosis that could not be performed with only the first ultrasound image displayed on the ultrasound diagnostic apparatus 10 by using the second ultrasound image.

As another example, the second image processing is image processing of generating an image for measurement, and the second ultrasound image is the image for measurement. The image processing apparatus 12 executes the second image processing, whereby the user (for example, a doctor) of the image processing apparatus 12 can acquire a measurement result from the second ultrasound image. For example, the user such as a doctor can perform measurement that could not be performed with only the first ultrasound image displayed on the ultrasound diagnostic apparatus 10 by using the second ultrasound image.

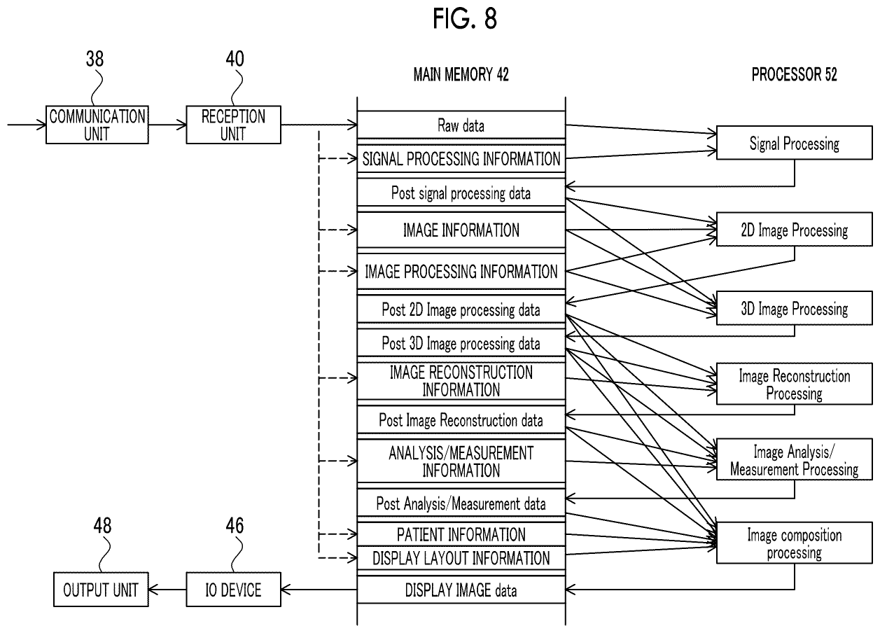

Hereinafter, a specific example of the first image processing executed by the processor 52 of the image processing apparatus 12 will be described with reference to FIG. 8. In FIG. 8, a solid line indicates a flow of signal data (for example, Raw data), and a broken line indicates a flow of control data.

In a case in which the information transmission data is transmitted as information from the ultrasound diagnostic apparatus 10 to the image processing apparatus 12, the second communication unit 38 receives the information transmission data as information. The reception unit 40 receives the information transmission data from the second communication unit 38 and stores the Raw data and the header data (US header data and IP header data) in the main memory 42. In a case in which the Raw data is compressed, the reception unit 40 develops (expands) the compressed data. The reception unit 40 may convert a format of the Raw data.

The signal processing information, the image information, the image processing information, the image reconstruction information, the analysis/measurement information, the patient information, and the display layout information are information (an example of the first information) included in the US header data added to the Raw data, and are stored in the main memory 42.

For example, the processor 52 executes, on the raw data, signal processing, 2D image processing, 3D image processing, image reconstruction processing, image analysis/measurement processing, and image composition processing. These kinds of processing are examples, and only a part of these kinds of processing may be executed.

The processor 52 reads the Raw data from the main memory 42 and applies the signal processing indicated by the signal processing information to the Raw data (signal processing). As a result, post signal processing data is generated and stored in the main memory 42.

In a case in which the processor 52 reads the post signal processing data, the image information, and the image processing information from the main memory 42 and applies two-dimensional image processing to the post signal processing data (2D image processing), post 2D image processing data is generated and stored in the main memory 42. The image information includes geometric information and three-dimensional structure information.

In a case in which the processor 52 reads the post signal processing data, the image information, and the image processing information from the main memory 42 and applies three-dimensional image processing to the post signal processing data (3D image processing), post 3D image processing data is generated and stored in the main memory 42.

In a case in which the processor 52 reads the post 2D image processing data, the post 3D image processing data, and the image reconstruction information from the main memory 42 and executes the reconstruction processing (image reconstruction processing), post image reconstruction data is generated and stored in the main memory 42.

In a case in which the processor 52 reads the post 2D image processing data, the post 3D image processing data, the post image reconstruction data, and the analysis/measurement information from the main memory 42 and executes the analysis processing (image analysis/measurement processing), post analysis/measurement data is generated and stored in the main memory 42.

In a case in which the processor 52 reads the post 2D image processing data, the post 3D image processing data, the post image reconstruction data, the post analysis/measurement data, the patient information, and the display layout information from the main memory 42 and executes the composition processing (image composition processing), display image data is generated and stored in the main memory 42. The display image data is output to the output unit 48 via the I/O device 46 and displayed on the display of the output unit 48. Data to be combined may be selected by the user, and the data selected by the user may be combined.

In a case in which information (an example of the first information) included in the US header data is used as the signal processing information, the image information, the image processing information, the image reconstruction information, the analysis/measurement information, the patient information, and the display layout information, the first ultrasound image is generated. For example, the first ultrasound image is displayed on the display of the output unit 48.

The processing shown in FIG. 8 is merely an example, and the processing to be executed is changed depending on, for example, the diagnosis site, the analysis, the measurement, the patient, the medical department, or the like.

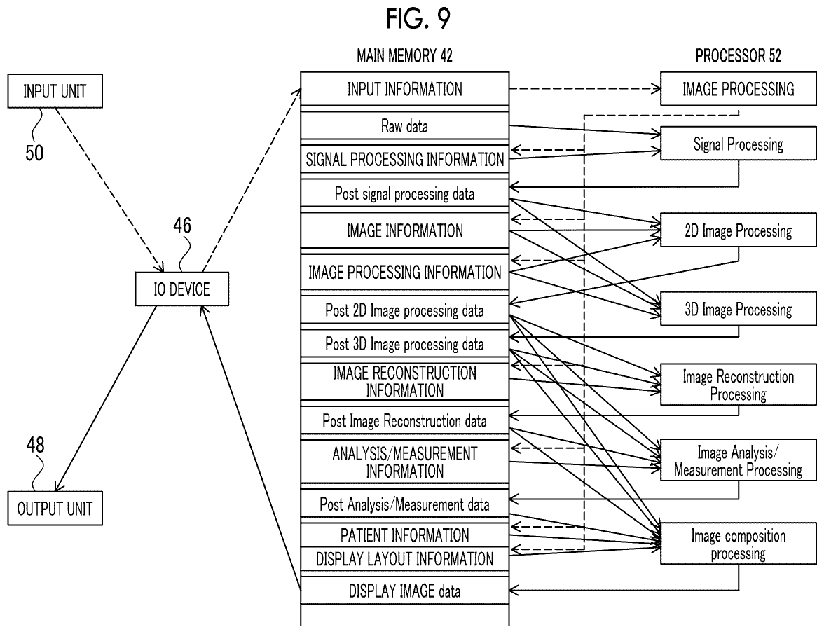

Hereinafter, a specific example of the second image processing executed by the processor 52 of the image processing apparatus 12 will be described with reference to FIG. 9. In FIG. 8, a solid line indicates a flow of signal data (for example, Raw data), and a broken line indicates a flow of control data.

The input information is stored in the main memory 42. The input information includes information indicating the second image processing or the like. The input information is input to the image processing apparatus 12 from the input unit 50 of the image processing apparatus 12 via the IO device 46 and is stored in the main memory 42. The input information may be received as information by the second communication unit 38 and input to the image processing apparatus 12.

The Raw data stored in the main memory 42 is data transmitted as information from the ultrasound diagnostic apparatus 10 to the image processing apparatus 12. In a case in which the information transmission data including the Raw data is transmitted as information from the ultrasound diagnostic apparatus 10 to the image processing apparatus 12, the Raw data stored in the main memory 42 is the data included in the information transmission data.

The signal processing information, the image information, the image processing information, the image reconstruction information, the analysis/measurement information, the patient information, and the display layout information are information (an example of the first information) included in the input information, and are stored in the main memory 42. For example, the user of the image processing apparatus 12 operates the input unit 50 to input each information included in the input information.

For example, the processor 52 executes, on the raw data, signal processing, 2D image processing, 3D image processing, image reconstruction processing, image analysis/measurement processing, and image composition processing. These kinds of processing are examples, and only a part of these kinds of processing may be executed.

The processor 52 reads the Raw data from the main memory 42 and applies the signal processing indicated by the signal processing information to the Raw data (signal processing). As a result, post signal processing data is generated and stored in the main memory 42.

In a case in which the processor 52 reads the post signal processing data, the image information, and the image processing information from the main memory 42 and applies two-dimensional image processing to the post signal processing data (2D image processing), post 2D image processing data is generated and stored in the main memory 42. The image information includes geometric information and three-dimensional structure information.

In a case in which the processor 52 reads the post signal processing data, the image information, and the image processing information from the main memory 42 and applies three-dimensional image processing to the post signal processing data (3D image processing), post 3D image processing data is generated and stored in the main memory 42.

In a case in which the processor 52 reads the post 2D image processing data, the post 3D image processing data, and the image reconstruction information from the main memory 42 and executes the reconstruction processing (image reconstruction processing), post image reconstruction data is generated and stored in the main memory 42.

In a case in which the processor 52 reads the post 2D image processing data, the post 3D image processing data, the post image reconstruction data, and the analysis/measurement information from the main memory 42 and executes the analysis processing (image analysis/measurement processing), post analysis/measurement data is generated and stored in the main memory 42.

In a case in which the processor 52 reads the post 2D image processing data, the post 3D image processing data, the post image reconstruction data, the post analysis/measurement data, the patient information, and the display layout information from the main memory 42 and executes the composition processing (image composition processing), display image data is generated and stored in the main memory 42. The display image data is output to the output unit 48 via the I/O device 46 and displayed on the display of the output unit 48. Data to be combined may be selected by the user, and the data selected by the user may be combined.

In a case in which information included in the input information input via the input unit 50 is used as the signal processing information, the image information, the image processing information, the image reconstruction information, the analysis/measurement information, the patient information, and the display layout information, the second ultrasound image is generated. For example, the second ultrasound image is displayed on the display of the output unit 48.

The processing shown in FIG. 9 is merely an example, and the processing to be executed is changed depending on, for example, the diagnosis site, the analysis, the measurement, the patient, the medical department, or the like.

Hereinafter, specific examples of the first ultrasound image and the second ultrasound image will be described.

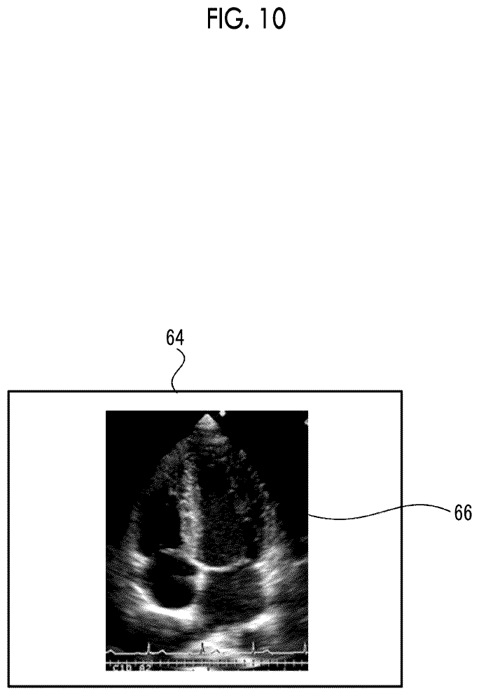

The ultrasound image displayed on the ultrasound diagnostic apparatus 10 will be described with reference to FIG. 10. FIG. 10 shows the first ultrasound image.

A screen 64 is displayed on the display of the display unit 24 of the ultrasound diagnostic apparatus 10. An ultrasound image 66 is the first ultrasound image and is displayed on the screen 64. The image processing unit 20 of the ultrasound diagnostic apparatus 10 generates the ultrasound image 66 by applying the first image processing to the Raw data (hereinafter, for convenience of description, referred to as “Raw data A”). For example, the first image processing may be image processing designated by the user (for example, an examination technician) of the ultrasound diagnostic apparatus 10, or may be image processing registered in advance in an examination protocol.

For example, the ultrasound image 66 is an image obtained by a cardiac apex approach and is a four-chamber cross-sectional image showing a cardiac apex. The four-chamber cross-sectional image is merely an example of the first ultrasound image, and another cross-sectional image, a Doppler image, a color Doppler image, or the like may be acquired and displayed as the first ultrasound image.

The first information indicating the first image processing for generating the ultrasound image 66 is included in the US header data added to the Raw data A for generating the ultrasound image 66. The information transmission data including the US header data and the Raw data A is transmitted as information from the ultrasound diagnostic apparatus 10 to the image processing apparatus 12. The image processing unit 54 of the image processing apparatus 12 generates the ultrasound image 66 by applying the first image processing indicated by the first information to the Raw data A. As a result, the ultrasound image 66 is reproduced by the image processing apparatus 12.

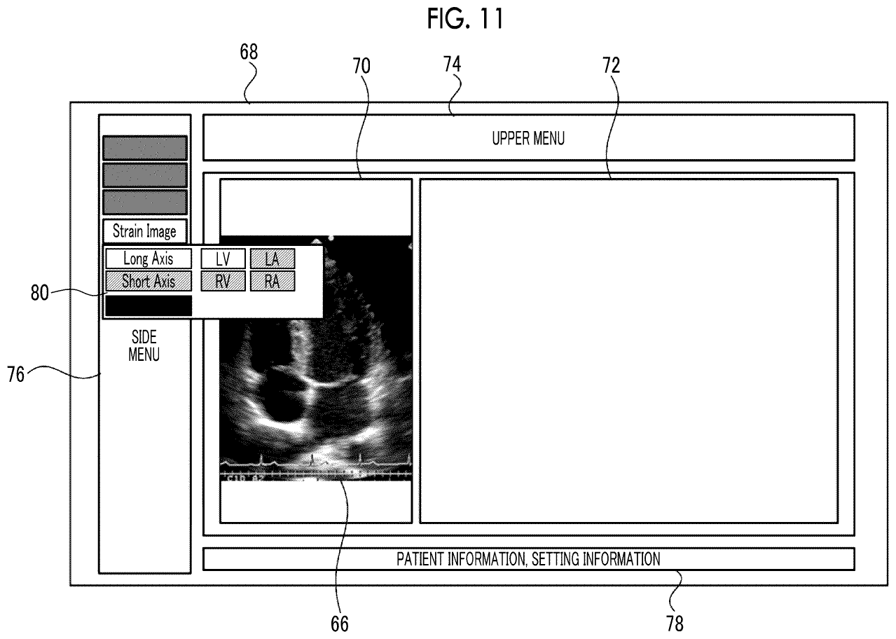

The ultrasound image displayed on the image processing apparatus 12 will be described with reference to FIG. 11. FIG. 11 shows a screen displayed on the image processing apparatus 12.

A screen 68 is displayed on the display of the output unit 48 of the image processing apparatus 12. The screen 68 includes a first display region 70 and a second display region 72. The first display region 70 is a region in which the first ultrasound image is displayed. The second display region 72 is a region in which the second ultrasound image is displayed. The first display region 70 and the second display region 72 are disposed side by side on the screen 68. As a result, the first ultrasound image and the second ultrasound image are displayed side by side on the screen 68.

For example, the image processing unit 54 of the image processing apparatus 12 generates the first ultrasound image by applying the first image processing indicated by the first information to the Raw data (Raw data included in the information transmission data) as information transmitted from the ultrasound diagnostic apparatus 10. The first information is included in the US header data added to the Raw data. As a result, the first ultrasound image generated by the ultrasound diagnostic apparatus 10 is reproduced by the image processing apparatus 12.

For example, the image processing unit 54 generates the ultrasound image 66 as the first ultrasound image by applying the first image processing indicated by the first information to the Raw data A. The first information is included in the US header data added to the Raw data A. As a result, the ultrasound image 66 is reproduced by the image processing apparatus 12.

As shown in FIG. 11, the display controller 56 of the image processing apparatus 12 displays the ultrasound image 66 in the first display region 70. As a result, the user (for example, a doctor) of the image processing apparatus 12 can observe the ultrasound image 66 displayed on the ultrasound diagnostic apparatus 10.

Further, an upper menu 74 and a side menu 76 are displayed on the screen 68. For example, the upper menu 74 and the side menu 76 include items such as analysis and measurement for the ultrasound image. In a case in which the user selects the desired analysis, measurement, or the like from the upper menu 74 or the side menu 76, the image processing unit 54 generates the second ultrasound image by applying the second image processing for realizing the selected analysis, measurement, or the like to the Raw data. The second ultrasound image is an ultrasound image for realizing the analysis, measurement, or the like. The display controller 56 displays the second ultrasound image in the second display region 72.

Further, as indicated by reference numeral 78, patient information or setting information is displayed on the screen 68. For example, the patient information is information included in the US header data. The setting information is information indicating settings for image processing.

In the example shown in FIG. 11, myocardial strain imaging (strain image), which is one of ultrasound imaging methods, is included in the side menu 76. The myocardial strain imaging is a method of imaging a change in movement of the myocardium and evaluating wall movement due to myocardial ischemia. For example, in a case in which the myocardial strain imaging is selected from the side menu 76, a separate menu 80 for the myocardial strain imaging is also displayed on the screen 68. The separate menu 80 includes items such as a cross section to be analyzed or measured. The user can designate a cross section or the like to be analyzed or measured by selecting an item included in the separate menu 80. The image processing unit 54 applies the second image processing to the Raw data in accordance with the item selected by the user. Of course, the myocardial strain imaging is merely an example, and other imaging methods, analyses, measurements, and the like may be included in the side menu 76.

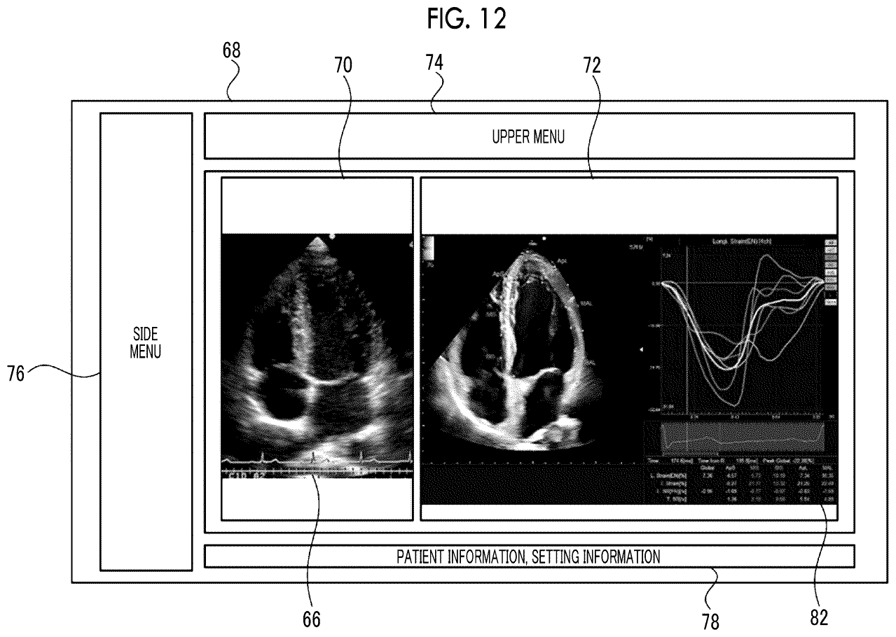

An ultrasound image 82 as the second ultrasound image is shown in FIG. 12. The image processing unit 54 generates the ultrasound image 82 by applying the second image processing corresponding to the analysis or measurement selected by the user to the Raw data A. The display controller 56 displays the ultrasound image 82 in the second display region 72.

The ultrasound image 82 is an image for myocardial strain imaging measurement. For example, the ultrasound image 82 is an image that represents a myocardial strain image of the four-chamber cross-sectional image acquired by the cardiac apex approach and a result of local strain measurement.

The ultrasound image 66, which is the first ultrasound image, and the ultrasound image 82, which is the second ultrasound image, are images generated from the same Raw data A. The ultrasound image 66 is an image similar to the ultrasound image displayed on the ultrasound diagnostic apparatus 10.

The user can perform the diagnosis, examination, or the like by comparing the first ultrasound image with the second ultrasound image. For example, the user can perform the diagnosis, examination, or the like by comparing the ultrasound image 66, which is the four-chamber cross-sectional image acquired by the cardiac apex approach, with the ultrasound image 82, which represents the result of the myocardial strain imaging measurement.

For example, the myocardial strain imaging measurement function is a function that is not possessed by the ultrasound diagnostic apparatus 10 (that is, a function that is realized by image processing that is not realized by the ultrasound diagnostic apparatus 10). The image processing apparatus 12 executes the myocardial strain imaging measurement, whereby myocardial strain imaging measurement that could not be realized only with the first ultrasound image displayed on the ultrasound diagnostic apparatus 10 can be realized by the image processing apparatus 12.

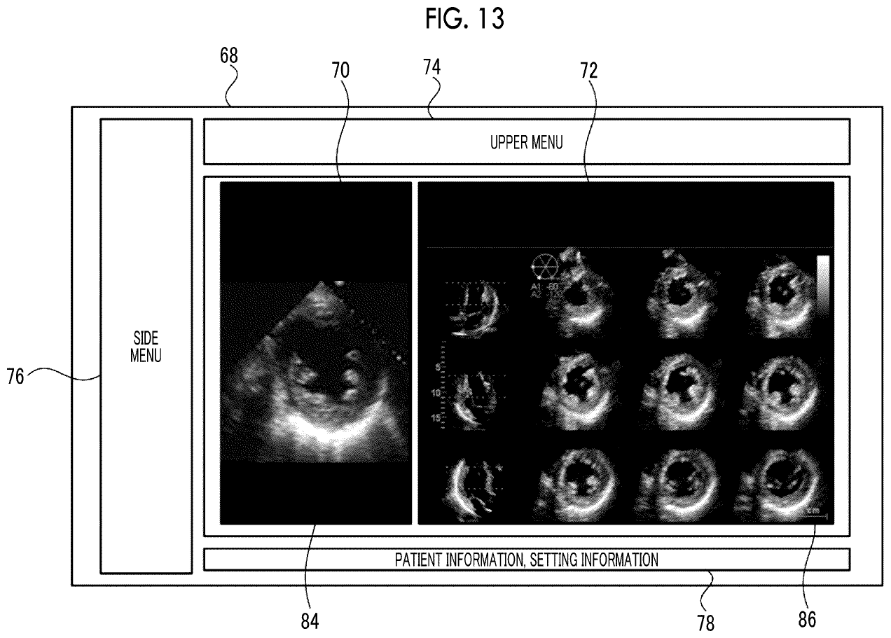

Another display example of the ultrasound image is shown in FIG. 13. For example, an ultrasound image 84 is displayed in the first display region 70, and an ultrasound image 86 is displayed in the second display region 72.

The ultrasound image 84 is an example of a first ultrasound image generated based on certain Raw data (referred to as “Raw data B” for convenience of description). That is, the ultrasound image 84 is an image generated by applying the first image processing to the Raw data B. The ultrasound image 84 is an image displayed on the ultrasound diagnostic apparatus 10.

The ultrasound image 86 is an example of a second ultrasound image generated based on the Raw data B. That is, the ultrasound image 86 is an image generated by applying the second image processing to the Raw data B.

For example, the ultrasound image 84 is an image representing one minor axis cross section of the heart. The one minor axis cross section may be designated by the user (for example, an examination technician) of the ultrasound diagnostic apparatus 10, or may be registered in advance in the examination protocol. The ultrasound image 86 is an image representing a plurality of minor axis cross sections of the same heart. The ultrasound image 86 includes a plurality of images. Each image of the plurality of images represents a separate minor axis cross section. For example, in a case in which the image processing for generating the images representing the plurality of minor axis cross sections is selected as the second image processing by the user (for example, a doctor) of the image processing apparatus 12, the image processing unit 54 generates the ultrasound image 86 by applying the image processing to the Raw data B.

For example, the user can perform the diagnosis, examination, or the like by comparing the ultrasound image 84 representing a certain minor axis cross section of the heart with the ultrasound image 86 representing a plurality of minor axis cross sections of the same heart.

For example, the function of generating the ultrasound image representing the plurality of minor axis cross sections is a function that is not possessed by the ultrasound diagnostic apparatus 10 (that is, a function that is realized by image processing that is not realized by the ultrasound diagnostic apparatus 10). The image processing apparatus 12 executes the function, whereby diagnosis, examination, or the like that could not be realized only with the first ultrasound image displayed on the ultrasound diagnostic apparatus 10 can be realized by the image processing apparatus 12.

In the examples shown in FIGS. 11 to 13, the display controller 56 displays the first ultrasound image and the second ultrasound image side by side on the screen 68. This is merely one of display examples, and the display controller 56 may display the first ultrasound image and the second ultrasound image on separate screens rather than displaying the first ultrasound image and the second ultrasound image side by side on the screen 68. For example, in a case in which the user of the image processing apparatus 12 issues an instruction to display the first ultrasound image, the display controller 56 displays the first ultrasound image on the display of the output unit 48. In a case in which the user of the image processing apparatus 12 issues an instruction to display the first ultrasound image, the display controller 56 displays the second ultrasound image on the display of the output unit 48. The display controller 56 may switch the displayed ultrasound image between the first ultrasound image and the second ultrasound image in response to the user's switching instruction.

As an example different from the examples shown in FIGS. 11 and 12, the screen 68 may include the second display region 72 and may not include the first display region 70. In this case, the display controller 56 displays the ultrasound image 82 as the second ultrasound image in the second display region 72, and does not display the first ultrasound image. The same applies to the example shown in FIG. 13. The display controller 56 displays the ultrasound image 86 as the second ultrasound image in the second display region 72 and does not display the first ultrasound image. In this manner, the user can perform the diagnosis, examination, or the like by referring to the second ultrasound image different from the first ultrasound image.

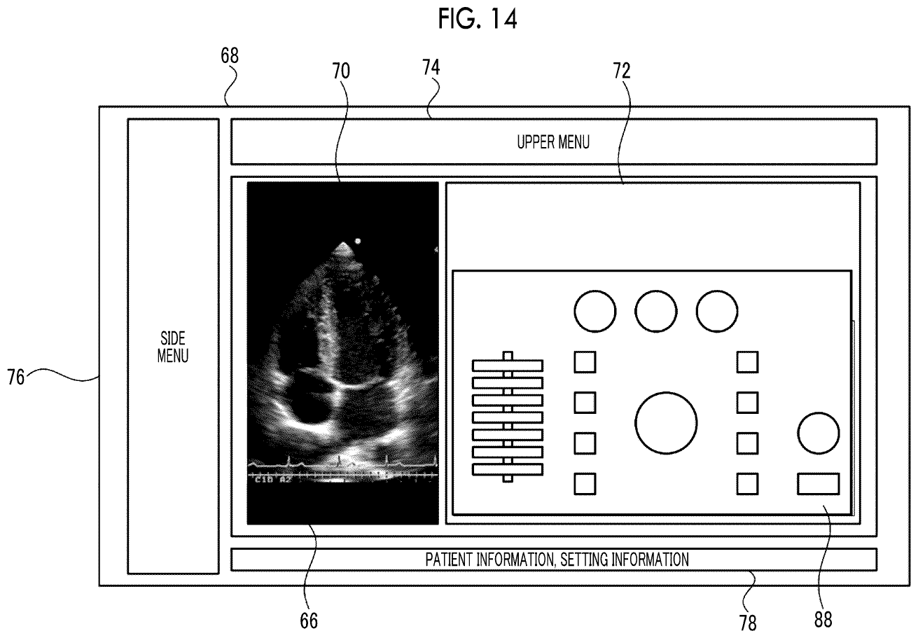

Another display example of the ultrasound image is shown in FIG. 14. The ultrasound image 66 as the first ultrasound image is displayed in the first display region 70. The display controller 56 displays a virtual console 88 in the second display region 72.

The virtual console 88 is an image that schematically represents an operation panel (for example, the operation panel included in the input unit 26) provided in the ultrasound diagnostic apparatus 10 and that represents a user interface for receiving input of a parameter for displaying an image used for the diagnosis or measurement. For example, the parameter is a gain, a time gain control (TGC), a focus, a depth of field, a function button, and the like.

For example, the virtual console 88 is an image representing a plurality of input devices such as a mouse, a trackball, a switch, a keyboard, a contact sensor, or haptics. Each input device represented by the virtual console 88 corresponds to each input device included in the input unit 26 of the ultrasound diagnostic apparatus 10.

The user operates the input device represented by the virtual console 88 to input a parameter for displaying the ultrasound image. The virtual console 88 receives the input.

For example, the image processing unit 54 applies the second image processing to the Raw data in accordance with the parameter input via the virtual console 88, thereby generating the second ultrasound image in which the parameter is reflected. The second ultrasound image is displayed on the screen 68.

The parameter input via the virtual console 88 may be transmitted as information from the image processing apparatus 12 to the ultrasound diagnostic apparatus 10. In this case, the ultrasound transmission/reception unit 16 of the ultrasound diagnostic apparatus 10 may transmit and receive the ultrasonic waves in accordance with the parameter, and the image processing unit 20 of the ultrasound diagnostic apparatus 10 may apply the first image processing to the Raw data in accordance with the parameter. For example, the user (for example, a doctor) of the image processing apparatus 12 can input the parameter by using the virtual console 88 while referring to the first ultrasound image displayed on the screen 68, thereby instructing the user (for example, an examiner) of the ultrasound diagnostic apparatus 10 to use the parameter or perform imaging.

In addition, the display controller 56 may display a parameter (for example, a gain) for generating the first ultrasound image on the screen 68 together with the first ultrasound image. As a result, the user of the image processing apparatus 12 can understand what kind of parameter or condition is used to capture the first ultrasound image.

As an example different from the example shown in FIG. 14, the screen 68 may include the second display region 72 and may not include the first display region 70. In this case, the display controller 56 displays the virtual console 88 in the second display region 72 and does not display the first ultrasound image. As a result, the user of the image processing apparatus 12 can use the virtual console 88 to instruct the user of the ultrasound diagnostic apparatus 10 to use the parameter or perform imaging.

The ultrasound diagnostic system according to the embodiment can be applied to, for example, point of care testing (POCT). For example, in a case in which a small and portable ultrasound diagnostic apparatus is used as the ultrasound diagnostic apparatus 10, the ultrasound examination can be performed in a hospital room, at a home, or the like. However, in general, the ultrasound diagnostic apparatus for POCT does not have the performance required for performing advanced diagnosis. For example, the ultrasound diagnostic apparatus for POCT does not have advanced image processing or a measurement function in some cases. With the ultrasound diagnostic system according to the embodiment, even in a case in which the ultrasound diagnostic apparatus 10 does not realize advanced image processing or a measurement function, the advanced image processing or the measurement function can be realized in a case in which the image processing apparatus 12 has the performance of performing the advanced image processing or the performance of realizing the measurement function.

In addition, the ultrasound diagnostic apparatus for POCT can be moved to various locations to acquire the ultrasound image. Therefore, the examiner can acquire the ultrasound image using the ultrasound diagnostic apparatus for POCT, and the doctor can use the image processing apparatus 12 to perform the signal processing, the image processing, the measurement, the analysis, and the like suitable for the diagnosis at a remote location. In this manner, it is possible to promote division of labor among medical professionals. For example, the ultrasound diagnostic system according to the embodiment may be used in home nursing care, emergency care, or the like.

The signal processing unit 18, the image processing unit 20, the display processing unit 22, the controller 28, the display controller 30, and the conversion unit 32 can be realized by using hardware resources such as a processor and an electronic circuit. A device such as a memory may be used as necessary for realizing the above-described configuration. The signal processing unit 18, the image processing unit 20, the display processing unit 22, the controller 28, the display controller 30, and the conversion unit 32 may be realized by, for example, a computer. That is, all or a part of the signal processing unit 18, the image processing unit 20, the display processing unit 22, the controller 28, the display controller 30, and the conversion unit 32 may be realized by cooperation between hardware resources, such as a central processing unit (CPU) or a memory included in a computer, and software (program) that defines the operation of the CPU or the like. The program is stored in the storage unit 36 of the ultrasound diagnostic apparatus 10 or other storage device through a recording medium, such as a CD or a DVD, or a communication path, such as a network. As another example, the signal processing unit 18, the image processing unit 20, the display processing unit 22, the controller 28, the display controller 30, and the conversion unit 32 may be realized by a digital signal processor (DSP), an application specific integrated circuit (ASIC), a field programmable gate array (FPGA), or the like. A graphics processing unit (GPU) or the like may be used. The signal processing unit 18, the image processing unit 20, the display processing unit 22, the controller 28, the display controller 30, and the conversion unit 32 may be realized by a single device. Each function of the signal processing unit 18, the image processing unit 20, the display processing unit 22, the controller 28, the display controller 30, and the conversion unit 32 may be realized by one or a plurality of devices.

The reception unit 40, the memory controller 44, the image processing unit 54, and the display controller 56 can be realized by using hardware resources such as a processor and an electronic circuit. A device such as a memory may be used as necessary for realizing the above-described configuration. The reception unit 40, the memory controller 44, the image processing unit 54, and the display controller 56 may be realized by, for example, a computer. That is, all or a part of the reception unit 40, the memory controller 44, the image processing unit 54, and the display controller 56 may be realized by cooperation between hardware resources, such as a central processing unit (CPU) or a memory included in a computer, and software (program) that defines the operation of the CPU or the like. The program is stored in the main memory 42 of the image processing apparatus 12 or another storage device through a recording medium, such as a CD or a DVD, or a communication path, such as a network. As another example, the reception unit 40, the memory controller 44, the image processing unit 54, and the display controller 56 may be realized by a digital signal processor (DSP), an application specific integrated circuit (ASIC), a field programmable gate array (FPGA), or the like. A graphics processing unit (GPU) or the like may be used. The reception unit 40, the memory controller 44, the image processing unit 54, and the display controller 56 may be realized by a single device. Each function of the reception unit 40, the memory controller 44, the image processing unit 54, and the display controller 56 may be realized by one or a plurality of devices.

What is claimed is:

1. An ultrasound diagnostic system comprising:
an ultrasound diagnostic apparatus; and
an image processing apparatus,
wherein the ultrasound diagnostic apparatus includes
an acquisition unit that acquires Raw data by transmitting and receiving ultrasonic waves,
a first image processing unit that generates a first ultrasound image by applying first image processing to the Raw data,
a first display controller that displays the first ultrasound image generated by the first image processing unit on a first display of the ultrasound diagnostic apparatus, and
a first information transmission unit that transmits the Raw data, to which the first image processing is not applied by the first image processing unit, to the image processing apparatus as information, and
the image processing apparatus includes
a second information reception unit that receives the Raw data as information,
a second image processing unit that generates a second ultrasound image different from the first ultrasound image by applying second image processing different from the first image processing to the Raw data, and
a second display controller that displays the second ultrasound image generated by the second image processing unit on a second display of the image processing apparatus.

2. The ultrasound diagnostic system according to claim 1,
wherein the second image processing is image processing that is not realized by the ultrasound diagnostic apparatus.

3. The ultrasound diagnostic system according to claim 1,
wherein the second image processing is image processing of generating an image for which a higher level of calculation capability than in the first image processing is required.

4. The ultrasound diagnostic system according to claim 3,
wherein the second image processing is image processing of generating an image for measurement, and
the second ultrasound image is the image for measurement.

5. The ultrasound diagnostic system according to claim 1,
wherein the second display controller displays, on the second display, an image that schematically represents an operation panel provided in the ultrasound diagnostic apparatus and that represents a user interface for receiving input of a parameter for displaying an image used for diagnosis or measurement.

6. The ultrasound diagnostic system according to claim 1,
wherein the ultrasound diagnostic apparatus is a portable ultrasound diagnostic apparatus.

7. An image processing apparatus comprising:
an information reception unit that receives Raw data as information from an ultrasound diagnostic apparatus that acquires the Raw data through transmission and reception of ultrasonic waves and generates a first ultrasound image by applying first image processing to the Raw data;
an image processing unit that generates a second ultrasound image different from the first ultrasound image by applying second image processing different from the first image processing to the Raw data; and
a display controller that displays the second ultrasound image generated by the image processing unit on a display.

8. An ultrasound diagnostic system comprising:
an ultrasound diagnostic apparatus; and
an image processing apparatus,
wherein the ultrasound diagnostic apparatus includes
an acquisition unit that acquires Raw data by transmitting and receiving ultrasonic waves,
a first image processing unit that generates a first ultrasound image by applying first image processing to the Raw data,
a first display controller that displays the first ultrasound image generated by the first image processing unit on a first display of the ultrasound diagnostic apparatus, and
a first information transmission unit that transmits the Raw data, to which the first image processing is not applied by the first image processing unit, and first information indicating the first image processing in association with each other to the image processing apparatus as information, and
the image processing apparatus includes
a second information reception unit that receives the Raw data and the first information, which are associated with each other, as information,
a second image processing unit that generates the first ultrasound image by applying the first image processing indicated by the first information to the Raw data and generates a second ultrasound image different from the first ultrasound image by applying second image processing different from the first image processing to the Raw data, and
a second display controller that displays the first ultrasound image and the second ultrasound image generated by the second image processing unit on a second display of the image processing apparatus.

9. The ultrasound diagnostic system according to claim 8,
wherein the second image processing is image processing that is not realized by the ultrasound diagnostic apparatus.

10. The ultrasound diagnostic system according to claim 8,
wherein the second image processing is image processing of generating an image for which a higher level of calculation capability than in the first image processing is required.

11. The ultrasound diagnostic system according to claim 10,
wherein the second image processing is image processing of generating an image for measurement, and
the second ultrasound image is the image for measurement.

12. The ultrasound diagnostic system according to claim 8,
wherein the second display controller displays the first ultrasound image and the second ultrasound image generated by the second image processing unit side by side on the second display.

13. The ultrasound diagnostic system according to claim 8,
wherein the second display controller displays, on the second display, an image that schematically represents an operation panel provided in the ultrasound diagnostic apparatus and that represents a user interface for receiving input of a parameter for displaying an image used for diagnosis or measurement.

14. The ultrasound diagnostic system according to claim 8,
wherein the ultrasound diagnostic apparatus is a portable ultrasound diagnostic apparatus.

15. An image processing apparatus comprising:
a second information reception unit that receives Raw data and first information indicating first image processing, which are associated with each other, as information from an ultrasound diagnostic apparatus that acquires the Raw data through transmission and reception of ultrasonic waves and generates a first ultrasound image by applying the first image processing to the Raw data;
a second image processing unit that generates the first ultrasound image by applying the first image processing indicated by the first information to the Raw data and generates a second ultrasound image different from the first ultrasound image by applying second image processing different from the first image processing to the Raw data; and
a second display controller that displays the first ultrasound image and the second ultrasound image generated by the second image processing unit side by side on a display.
